(19) United States
(12) Patent Application Publication
(10) Pub. No.: US 2009/0131939 A1
(43) Pub. Date: May 21, 2009

(54) INFLATABLE MOLD FOR MAINTAINING POSTERIOR SPINAL ELEMENTS IN A DESIRED ALIGNMENT

(75) Inventors: Michael Ahrens, Neustadt i.H. (DE); Erik O. Martz, Savage, MN (US); Ronald Burke, Deep Haven, MN (US); Benjamin F. Carter, Eden Prairie, MN (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: Disc Dynamics, Inc., Eden Prairie, MN (US)

(21) Appl. No.: 12/338,544

(22) Filed: Dec. 18, 2008

Related U.S. Application Data
(63) Continuation-in-part of application No. 11/420,055, filed on May 24, 2006.
Application No. 11/420,055 is a continuation-in-part of application No. 12/203,727, filed on Sep. 3, 2008.

(51) Int. Cl. A61B 17/00; A61B 17/58; B28B 7/32; A61F 2/44

(52) U.S. Cl. 606/80; 606/300; 264/314; 425/2; 623/17.11

(57) ABSTRACT

A system for the in situ formation of prostheses between adjacent vertebrae of a patient. The system includes a first mold adapted to be located in an intervertebral disc space between the adjacent vertebrae and at least a second mold adapted to be positioned between adjacent posterior elements. Lumens are fluidly coupled to each of the molds. One or more in situ curable biomaterials are delivered through the lumens to the molds. The at least partially cured biomaterial and the molds cooperate to maintain a desired alignment and spacing of the intervertebral disc space and the posterior elements.

The present application is a Continuation-In-Part of U.S. application Ser. No. 11/420,055, entitled Mold Assembly for Intervertebral Prosthesis, filed May 24, 2006 and a Continuation-In-Part of U.S. application Ser. No. 12/203,727, entitled Retention Structure for In-Situ Formation of Intervertebral Prosthesis, filed Sep. 3, 2008, which are hereby incorporated by reference.

### FIELD OF THE INVENTION

The present invention relates to various mold assemblies for forming an intervertebral prosthesis in situ, and in particular to molds for an intervertebral disc space and posterior spinal elements adapted to receive an in situ curable biomaterial and a method of filling the mold.

### BACKGROUND OF THE INVENTION

The intervertebral discs, which are located between adjacent vertebrae in the spine, provide structural support for the spine as well as the distribution of forces exerted on the spinal column. An intervertebral disc consists of three major components: cartilage endplates, nucleus pulposus, and annulus fibrosus.

In a healthy disc, the central portion, the nucleus pulposus or nucleus, is relatively soft and gelatinous; being composed of about 70 to 90% water. The nucleus pulposus has high proteoglycan content and contains a significant amount of Type II collagen and chondrocytes. Surrounding the nucleus is the annulus fibrosus, which has a more rigid consistency and contains an organized fibrous network of approximately 40% Type I collagen, 60% Type II collagen, and fibroblasts. The annular portion serves to provide peripheral mechanical support to the disc, afford torsional resistance, and contain the softer nucleus while resisting its hydrostatic pressure.

Intervertebral discs, however, are susceptible to disease, injury, and deterioration during the aging process. Disc herniation occurs when the nucleus begins to extrude through an opening in the annulus, often to the extent that the herniated material impinges on nerve roots in the spine or spinal cord. The posterior and posterolateral portions of the annulus are most susceptible to attenuation or herniation, and therefore, are more vulnerable to hydrostatic pressures exerted by vertical compressive forces on the intervertebral disc. Various injuries and deterioration of the intervertebral disc and annulus fibrosus are discussed by Osti et al., Annular Tears and Disc Degeneration in the Lumbar Spine, J. Bone and Joint Surgery, 74-B(5), (1982) pp. 678-682; Osti et al., Annulus Tears and Intervertebral Disc Degeneration, Spine, 15(8) (1990) pp. 762-767; Kamblin et al., Development of Degenerative Spondylosis of the Lumbar Spine after Partial Discectomy, Spine, 20(5) (1995) pp. 599-607.

Many treatments for intervertebral disc injury have involved the use of nuclear prostheses or disc spacers. A variety of prosthetic nuclear implants are known in the art. For example, U.S. Pat. No. 5,047,055 (Bao et al.) teaches a swellable hydrogel prosthetic nucleus. Other devices known in the art, such as intervertebral spacers, use wedges between vertebrae to reduce the pressure exerted on the disc by the spine. Intervertebral disc implants for spinal fusion are known in the art as well, such as disclosed in U.S. Pat. Nos. 5,425,772 (Brantigan) and 4,834,757 (Brantigan).

Further approaches are directed toward fusion of the adjacent vertebrate, e.g., using a cage in the manner provided by Sulzer. Sulzer's BAK® Interbody Fusion System involves the use of hollow, threaded cylinders that are implanted between two or more vertebrae. The implants are packed with bone graft to facilitate the growth of vertebral bone. Fusion is achieved when adjoining vertebrae grow together through and around the implants, resulting in stabilization.

Apparatuses and/or methods intended for use in disc repair have also been described for instance in French Patent Appl. No. FR 2 639 823 (Garcia) and U.S. Pat. No. 6,187,048 (Milner et al.). Both references differ in several significant respects from each other and from the apparatus and method described below.

Prosthetic implants formed of biomaterials that can be delivered and cured in situ, using minimally invasive techniques to form a prosthetic nucleus within an intervertebral disc have been described in U.S. Pat. Nos. 5,556,429 (Felt) and 5,888,220 (Felt et al.), and U.S. Patent Publication No. US 2003/0195628 (Felt et al.), the disclosures of which are incorporated herein by reference. The disclosed method includes, for instance, the steps of inserting a collapsed mold apparatus (which in a preferred embodiment is described as a “mold”) through an opening within the annulus, and filling the mold to the point that the mold material expands with a flowable biomaterial that is adapted to cure in situ and provide a permanent disc replacement. Related methods are disclosed in U.S. Pat. No. 6,224,630 (Bao et al.), entitled “Implantable Tissue Repair Device” and U.S. Pat. No. 6,079,868 (Rydell), entitled “Static Mixer”, the disclosures of which are incorporated herein by reference.

Intervertebral implants comprising a spacer that is inserted between the spinous processes are currently used to stabilize the spine, distract or increase the opening in the foramen, unload the intervertebral discs, and the like. Examples of such devices are shown in U.S. Pat. Nos. 7,306,628 (Zucherman et al.); 7,238,204 (Le Couedic et al.); 6,132,464 (Martin); and 5,498,262 (Bryan).

These spacers, generally made of titanium alloy, present a notch at each of their ends, with the spinous processes being received in the notches. In addition, the spacer is held by ties, interconnecting the two opposite edges of each of the notches and tightened around part of the wall of each spinous processes.

Such implants limit the extent to which the vertebrae can move towards each other since, when the spine is in extension, the spinous processes tend to come into abutment against the bottoms of the opposite notches in which they are inserted. However, the material of which the spacer is made is hard compared with the material of an intervertebral disk which, when it is intact, limits the extent to which the vertebrae can move towards each other, so much so that the jolts which can be transmitted to the spine, e.g. while walking, are not damped between two vertebrae interconnected by a spacer. Furthermore, since the spacer does not have the same mechanical properties as the remaining portion of the intervertebral disk, the overall mechanical properties of the spine present significant discontinuities compared with an intact spine, thereby increasing deterioration of the intervertebral disk.

U.S. Pat. No. 6,733,534 (Sherman) discloses a system and method of positioning a spacer within a patient. The spacer has a first form having a reduced size such that it can be inserted into the patient in a minimally invasive manner. Once inserted to an application point within the patient, the spacer is expanded to a desired size. In one embodiment, the spacer is constructed of a flexible material that is sized to fit within the opening in the patient and be delivered to the application point. Biomaterial is then fed into the spacer to expand the size to the desired dimensions.

FIG. 1 illustrates an exemplary prior art catheter 11 with mold or balloon 13 located on the distal end. In the illustrated embodiment, biomaterial 23 is delivered to the mold 13 through the catheter 11. Secondary tube 11′ evacuates air from the mold 13 before, during and/or after the biomaterial 23 is delivered. The secondary tube 11′ can either be inside or outside the catheter 11.

### BRIEF SUMMARY OF THE INVENTION

The present application is directed to a system for the in situ formation of prostheses between adjacent vertebrae of a patient. The system includes a mold assembly containing a partially cured biomaterial that maintains posterior spinal elements in a desired alignment. Posterior elements refers any of the spinous processes, transverse processes, anterior or posterior tubercle of transverse process, superior and inferior articular process, articular pillar, and facets. The mold assembly may also be used in combination with a variety of other spinal devices, including nucleus replacement, total disc replacement, interbody fusion, vertebral body replacement, pedicle screw fixation, and the like.

In one embodiment, the system includes a first mold adapted to be located in an intervertebral disc space between the adjacent vertebrae and at least a second mold adapted to be positioned between adjacent posterior elements. Lumens are fluidly coupled to each of the molds. One or more in situ curable biomaterials are delivered through the lumens to the molds. The at least partially cured biomaterial and the molds cooperate to maintain a desired alignment of the intervertebral disc space and the posterior elements. At least a second mold is adapted to be positioned between posterior elements on one side of a sagittal plane of the patient and a third mold adapted to be positioned between posterior elements on an opposite side of the sagittal plane. The posterior elements are optionally contoured to enhance the engagement with the second and third molds. In one embodiment, the quantity of biomaterial is adjusted to displace the posterior elements a greater amount on one side of the sagittal plane.

In another embodiment, a hole is drilled in a superior articulating inferior facet of an inferior vertebrae. A mold assembly is located in the hole and inflated with biomaterial. A head or bumper of the mold assembly abuts against inferior articulating facet of the superior vertebrae. In an alternate embodiment, a contour is formed on the inferior facet of the superior vertebrae so the head of the mold assembly engages with the contoured surface of the inferior facet.

In another embodiment, the mold assembly is located between the inferior articulating facet of the superior vertebrae and the superior articulating facet of the inferior vertebrae. In one embodiment, the mold assembly is inserted through hole in the inferior articulating facet of the superior vertebrae. A catheter segment extending above the inferior facet of the superior vertebrae can optionally be used to anchor the mold assembly to the facets. When the mold assembly is inflated with the biomaterial it pushes the inferior articulating facet and associated superior vertebrae upwards and distracts the foramen. The mold assemblies disclosed herein can be used alone or in combination with other embodiments of the mold assemblies.

One or more discrete reinforcing structures are optionally located in at least one of the molds. The reinforcing structure can be located inside or outside the interior cavity of the mold. The reinforcing structure can be one or more reinforcing bands extending around the mold, one or more collapsed structures adapted to be delivered through the lumen into the mold, a plurality of structures adapted to be delivered sequentially through the lumen into the mold, and the like. The reinforcing structures can be delivered through the lumen before, during or after delivery of the mold.

The reinforcing structure can be an expandable structure. The reinforcing structure can optionally include a plurality of independently positionable and/or interlocking members. The reinforcing structure preferably operates in both tension and compression.

In one embodiment, the reinforcing structure is a generally honeycomb structure. The honeycomb structure can be an expandable assembly or a plurality of discrete components.

A valve preferably fluidly couples the lumens to the molds. The lumens are preferably releasably coupled to the molds.

The present invention is also directed to an apparatus for the in-situ formation of a prosthesis between adjacent posterior elements of the spine. The mold is adapted to be positioned between the adjacent posterior elements. The mold including at least one interior cavity adapted to receive a flowable, curable biomaterial. At least one lumen is fluidly coupled to the mold. A valve assembly releasably couples the lumen to the mold. The flowable, curable biomaterial is adapted to be delivered through the at least one lumen to the mold. A biomaterial delivery apparatus preferably delivers the biomaterial through the lumen to expand the mold while the mold is located between the adjacent posterior elements. The at least partially cured biomaterial and the mold cooperate to maintain a desired alignment between the posterior elements.

In one embodiment, at least a portion of the mold includes a porous structure and/or a biodegradable material. In another embodiment, at least one reinforcing structure is located in the mold. The mold preferably comprises a predetermined shape. In one embodiment, the mold comprises a center portion with a plurality of extensions adapted to engage the posterior elements. In another embodiment, the mold includes an exterior surface adapted to facilitate tissue in-growth. The exterior surface may also include a bioactive agent and/or exterior surface textured to grip the posterior elements. In another embodiment, the mold includes porous structure containing a bioactive agent. The mold and the biomaterial are preferably delivered using minimally invasive techniques.

The present is also directed to a method for the in-situ formation of prostheses between adjacent vertebrae of a patient. A first mold is positioned in an intervertebral disc space between the adjacent vertebrae. At least a second mold is positioned between adjacent posterior elements. The method also includes positioning the second mold between posterior elements on one side of a sagittal plane of the patient and positioning a third mold between posterior elements on an opposite side of the sagittal plane. At least one lumen is fluidly coupled to each of the molds. A flowable, curable biomaterial is delivered through the lumens to the first and second molds. The first, second and third molds can be filled sequentially or simultaneously. The biomaterial is at least partially cured. The at least partially cured biomaterial maintains a desired alignment of the intervertebral disc space and the posterior elements.

The second and third molds can be located between any combination of the posterior elements, including the spinous processes, transverse processes, anterior or posterior tubercle of transverse process, superior and inferior articular process, articular pillar, and facets. In one embodiment, a greater quantity of curable biomaterial is delivered to the second mold than the third mold.

Minimally invasive refers to a surgical mechanism, such as microsurgical, percutaneous, or endoscopic or arthroscopic surgical mechanism. In one embodiment, the entire procedure is minimally invasive, for instance, through minimal incisions in the epidermis (e.g., incisions of less than about 6 centimeters, and more preferably less than 4 centimeters, and preferably less than about 2 centimeters), typically without the need to resect tissue in order to gain access to the application point. In another embodiment, the procedure is minimally invasive only with respect to the annular wall and/or pertinent musculature, or bony structure. Such surgical mechanism are typically accomplished by the use of visualization such as fiber optic or microscopic visualization, and provide a post-operative recovery time that is substantially less than the recovery time that accompanies the corresponding open surgical approach. Background on minimally invasive surgery can be found in German and Foley, Minimal Access Surgical Techniques in the Management of the Painful Lumbar Motion Segment, 30 SPINE 16S, n. S52-S59 (2005). Minimally invasive techniques are advantageous because they can be performed with the use of a local anesthesia, have a shorter recovery period, result in little to no blood loss, greatly decrease the chances of significant complications, and are generally less expensive.

Mold generally refers to the portion or portions of the present invention used to receive, constrain, shape and/or retain a flowable biomaterial in the course of delivering and curing the biomaterial in situ. A mold may include or rely upon natural tissues (such as the annular shell of an intervertebral disc or the end plates of the adjacent vertebrae) for at least a portion of its structure, conformation or function. For example, the mold may form a fully enclosed cavity or chamber or may rely on natural tissue for a portion thereof. The mold, in turn, is responsible, at least in part, for determining the position and final dimensions of the cured prosthetic implant. As such, its dimensions and other physical characteristics can be predetermined to provide an optimal combination of such properties as the ability to be delivered to a site using minimally invasive means, filled with biomaterial, control moisture contact, and optionally, then remain in place as or at the interface between cured biomaterial and natural tissue. In a particularly preferred embodiment the mold material can itself become integral to the body of the cured biomaterial.

The present mold will generally include both at least one cavity for the receipt of biomaterial and at least one lumen to that cavity. Multiple molds, either discrete or connected, may be used in some embodiments. Some or all of the material used to form the mold will generally be retained in situ, in combination with the cured biomaterial, while some or the entire lumen will generally be removed upon completion of the procedure. The mold and/or lumens can be biodegradable or bioresorbable. Examples of biodegradable materials can be found in U.S. Publication Nos. 2005-0197422; 2005-0238683; and 2006-0051394, the disclosures of which are hereby incorporated by reference. The mold can be an impermeable, semi-permeable, or permeable membrane. In one embodiment, the mold is a highly permeable membrane, such as for example a woven or non-woven mesh or other durable, loosely woven fabrics. The mold and/or biomaterial can include or be infused with drugs, pH regulating agents, pain inhibitors, and/or growth stimulants.

Biomaterial will generally refers to a material that is capable of being introduced to the site of a joint and cured to provide desired physical-chemical properties in vivo. In a preferred embodiment the term will refer to a material that is capable of being introduced to a site within the body using minimally invasive means, and cured or otherwise modified in order to cause it to be retained in a desired position and configuration. Generally such biomaterials are flowable in their uncured form, meaning they are of sufficient viscosity to allow their delivery through a lumen of on the order of about 1 mm to about 10 mm inner diameter, and preferably of about 2 mm to about 6 mm inner diameter. Such biomaterials are also curable, meaning that they can be cured or otherwise modified, in situ, at the tissue site, in order to undergo a phase or chemical change sufficient to retain a desired position and configuration.

The mold assembly of the present invention uses one or more discrete access points or annulotomies into the intervertebral disc space, and/or through the adjacent vertebrae. The annulotomies facilitate performance of the nuclectomy, imaging or visualization of the procedure, delivery of the biomaterial to the mold through one or more lumens, drawing a vacuum on the mold before, during and/or after delivery of the biomaterial, and securing the prosthesis in the intervertebral disc space during and after delivery of the biomaterial.

### BRIEF DESCRIPTION OF THE SEVERAL VIEWS OF THE DRAWING

FIG. 1 is an exemplary prior art catheter and mold.

FIG. 2 is a schematic illustration of various entry paths for use in accordance with the present invention.

FIGS. 3A and 3B are cross-sectional views of an annulus containing a mold assembly with one or more valves in accordance with the present invention.

FIGS. 3C and 3D are side sectional views of a mold assembly including a connector assembly in accordance with the present invention.

FIG. 3E is a cross-sectional view of the mold assembly of FIGS. 3C and 3D implanted in a patient.

FIGS. 4A and 4B are cross-sectional views of an annulus containing a mold assembly with an alternate valves in accordance with the present invention.

FIGS. 5A and 5B are cross-sectional views of an annulus containing a mold assembly with alternate valves in accordance with the present invention.

FIGS. 6A and 6B are cross-sectional views of an annulus containing a mold assembly with reinforcing bands in accordance with the present invention.

FIGS. 6C and 6D are cross-sectional views of an annulus containing a mold assembly comprising a reinforcing band in accordance with the present invention.

FIGS. 7A and 7B are cross-sectional views of an annulus containing a mold assembly containing an expandable reinforcing structure in accordance with the present invention.

FIG. 8 is a cross-sectional view of an annulus containing a mold assembly with an alternate expandable reinforcing structure in accordance with the present invention.

FIG. 9 is a cross-sectional view of an annulus containing a mold assembly with an alternate expandable reinforcing structure in accordance with the present invention.

FIGS. 10A and 10B are cross-sectional views of an annulus containing a mold assembly with a plurality of helical coils assembled into a reinforcing structure in accordance with the present invention.

FIGS. 11A and 11B are cross-sectional views of an annulus containing a mold assembly with a plurality of spherical reinforcing structures in accordance with the present invention.

FIG. 12 is a cross-sectional view of an annulus containing a mold assembly with an assembled reinforcing structure in accordance with the present invention.

FIG. 13 is a cross-sectional view of an annulus containing a mold assembly with an alternate assembled reinforcing structure in accordance with the present invention.

FIG. 14 is a cross-sectional view of an annulus containing a mold assembly with a fibrous reinforcing structure in accordance with the present invention.

FIG. 15A is a cross-sectional view of an annulus containing a mold assembly with an expandable honeycomb reinforcing structure in accordance with the present invention.

FIGS. 15B and 15C are side and top sectional views of an annulus containing a mold assembly with an alternate expandable honeycomb structure in accordance with the present invention.

FIG. 16 is a cross-sectional view of an annulus containing a mold assembly with multiple molds and a pressure activated reinforcing structure in accordance with the present invention.

FIGS. 17A and 17B are cross-sectional views of an annulus containing variations of the mold assembly of FIG. 16.

FIGS. 18A and 18B are cross-sectional views of an annulus containing a mold assembly with multiple molds and an alternate pressure activated reinforcing structure in accordance with the present invention.

FIG. 18C is a cross-sectional views of the mold assembly of FIGS. 18A and 18B used in a mono-portal application in accordance with the present invention.

FIGS. 19A and 19B are cross-sectional views of an annulus containing a mold assembly with patterned radiopaque markers in accordance with the present invention.

FIGS. 20A and 20B are cross-sectional views of an annulus containing a mold assembly with an alternate patterned radiopaque markers in accordance with the present invention.

FIG. 21 is cross-sectional views of an annulus containing a pair of nested molds in accordance with the present invention.

FIG. 22 is a perspective view of the present mold assembly separating adjacent transverse processes in accordance with the present invention.

FIG. 23 is a perspective view of the present mold assembly separating adjacent spinous processes in accordance with the present invention.

FIGS. 24-27 illustrate a mold assembly positioned to abut against an inferior articulating facet of a superior vertebrae in accordance with an embodiment of the present invention.

FIG. 28-30 illustrate a mold assembly positioned to abut against a contoured surface on an inferior articulating facet of a superior vertebrae in accordance with an embodiment of the present invention.

FIG. 31 illustrates a mold assembly located between adjacent facets in accordance with an embodiment of the present invention.

FIGS. 32A-32C illustrate alternate molds with extensions adapted to engage with posterior elements.

FIG. 33 is a perspective view of a mold assembly with multiple lumens in accordance with the present invention.

FIG. 34 is a perspective view of a mold assembly with a reinforcing structure in accordance with an embodiment of the present invention.

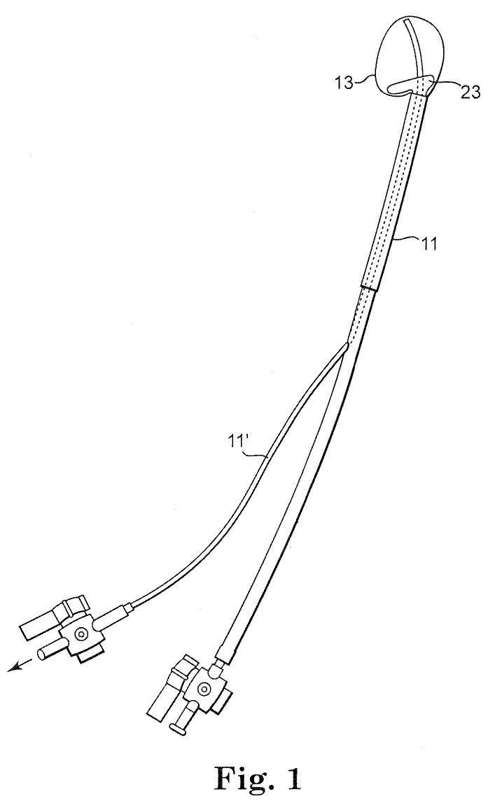

### DETAILED DESCRIPTION OF THE INVENTION

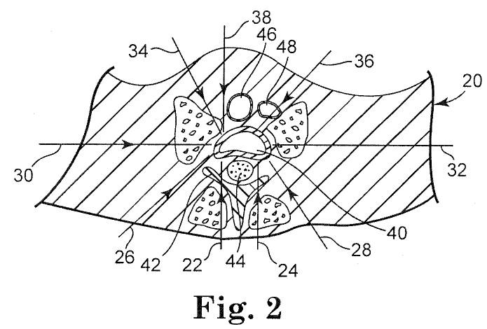

FIG. 2 is a cross-sectional view of a human body 20 showing various access paths 22 through 38 to the intervertebral disc 40 for performing the method of the present invention. The posterior paths 22, 24 extend either between superior and inferior transverse processes 42, or between the laminae (interlaminar path) on either side of the spinal cord 44. The posterolateral paths 26, 28 are also on opposite sides of the spinal cord 44 but at an angle of about 35-45 degrees relative to horizontal relative to the posterior paths 22, 24. The lateral paths 30, 32 extend through the side of the body. The anterior path 38 and anterolateral path 34 extend past the aorta iliac artery 46, while the anterolateral path 36 is offset from the inferior vena cava, iliac veins 48.

Depending on the disc level being operated on, and the patient anatomy. Generally, the aorta and vena cava split at the L4 vertebral body. At L5S1 the approach is typically a midline anterior approach. At L4/5 the approach may be either midline anterior or anterolateral, depending on the patient anatomy and how easy it is to retract the vessels. In some usages, the anterior approach is deemed a midline approach and the anterolateral approach is deemed an angled approach offset from the midline anterior approach.

The present method and apparatus use one or more of the access paths 22 through 38. While certain of the access paths 22 through 38 may be preferred depending on a number of factors, such as the nature of the procedure, any of the access paths can be used with the present invention.

In one embodiment, delivery catheter instruments are positioned along two or more of the access paths 22 through 38 to facilitate preparation of the intervertebral disc 40. Preparation includes, for example, formation of two or more annulotomies through the annular wall, removal of some or all of the nucleus pulposus to form a nuclear cavity, imaging of the annulus and/or the nuclear cavity, and positioning of the present multi-lumen mold in the nuclear cavity. In another embodiment, the present multi-lumen mold is positioned in the intervertebral disc 40 without use of delivery catheters.

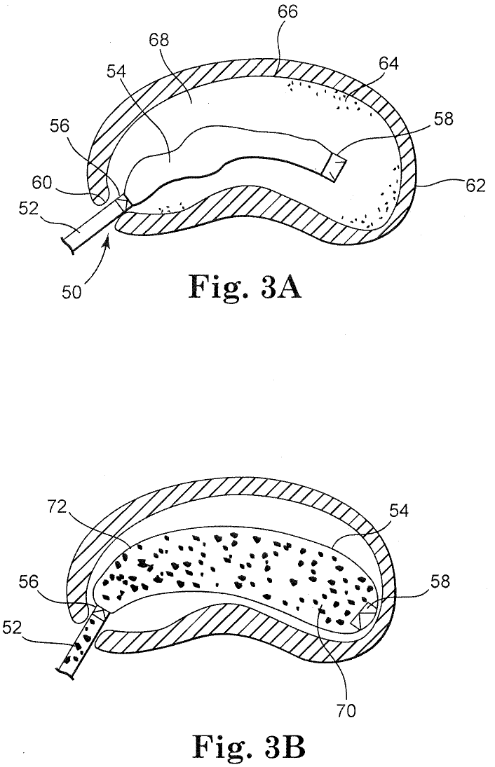

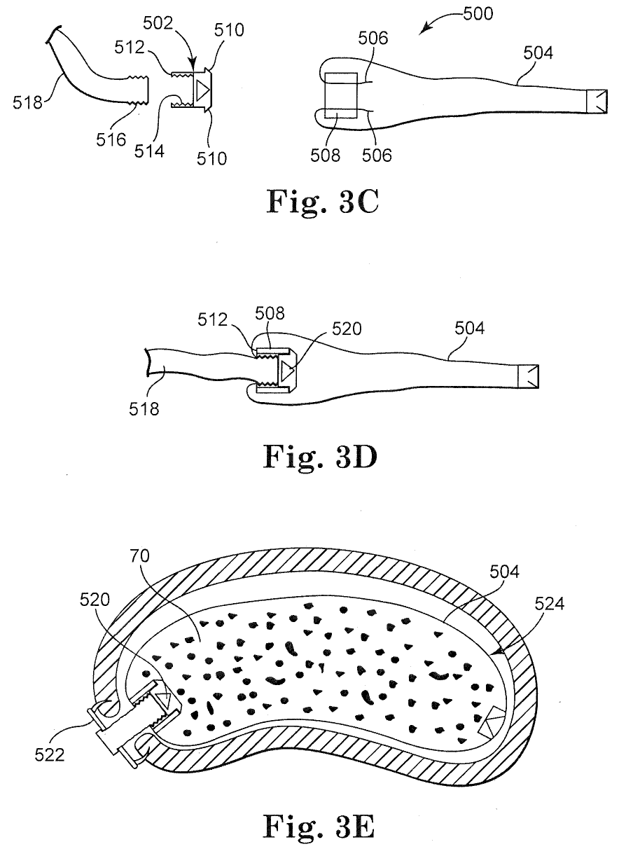

FIG. 3A illustrates one embodiment of a mold assembly 50 in accordance with the present invention. The mold assembly 50 includes lumen 52 fluidly coupled to mold 54. In the illustrated embodiment, valve 56 is provided at the interface between the lumen 52 and the mold 54. In one embodiment, valve 58 is optionally located at a separate location on the mold 54.

The method of using the present mold assembly 50 involves forming an annulotomy 60 at a location in the annulus 62. The nucleus pulposus 64 located in the disc space 66 is preferably substantially removed to create a nuclear cavity 68. As illustrated in FIG. 3A, some portion of the nucleus pulposus 64 may remain in the nuclear cavity 68 after the nuclectomy. The mold assembly 50 is then inserted through the annulotomy 60 so that the mold 54 is positioned in the nuclear cavity 68.

As illustrated in FIG. 3B, biomaterial 70 is delivered through the lumen 52 into the mold 54. As the biomaterial 70 progresses through the mold 54, at least a portion of the air located in the mold 54 is preferably pushed out through the valve 58. In the illustrated embodiment, the valves 56 and 58 are preferably check valves that are forced into the closed position by the pressure of the biomaterial 70. Once delivery of the biomaterial 70 is substantially completed, the lumen 52 is detached from the mold 54 removed from the annulotomy 60. In the illustrated embodiment, the valve 56 permits the lumen 52 to be separated and removed before the biomaterial 70 has cured.

In one embodiment, one or more of the mold 54, the valves 56, 58, and/or the lumens 52 have radiopaque properties that facilitate imaging of the prosthesis 72 being formed. In another embodiment, the lumen 52 is releasably attached to the valve 56 to facilitate removal.

In one embodiment, the lumen 52 is threaded to the valve 56. In another embodiment, a quick release interface is used to attach the lumen 52 to the valve 56.

FIGS. 3C and 3D are assembly views of a mold assembly 500 with a connection assembly 502 recessed in the mold 504 in accordance with the present invention. Open end 506 of the mold 504 is inserted into sleeve 508. The connector assembly 502 is then coupled to the sleeve 508. The open end 506 is secured between the sleeve 508 and connector assembly 502. In the illustrated embodiment, distal end of the connector assembly 502 includes a mechanical interface 510 that mechanically couples with the sleeve 508. The connector assembly 502 can be coupled to the open end 506 of the mold 504 and the sleeve 508 using a variety of techniques, such as adhesives, mechanical interlocks, fasteners, and the like.

The exposed end 512 of the connector assembly 502 preferably includes a mechanical interlock 514, such as for example internal threads, that couple with a corresponding interlock 516, such as external threads, on the lumen 518. As best illustrated in FIG. 3E, the biomaterial 70 is retained in the mold by valve 520 preferably located in the connector assembly 502. In the illustrated embodiment, the connector assembly 502 and/or the valve 520 are substantially flush with the outer surface of the mold 504. In another embodiment, the connector assembly 502 may protrude above the outer surface of the mold 504. The lumen 518 is preferably removed from the mold assembly 500 before the biomaterial 70 is cured. The exposed mechanical interlock 514 on the connector assembly 502 can optionally be used to attach a securing device 522 to the prosthesis 524.

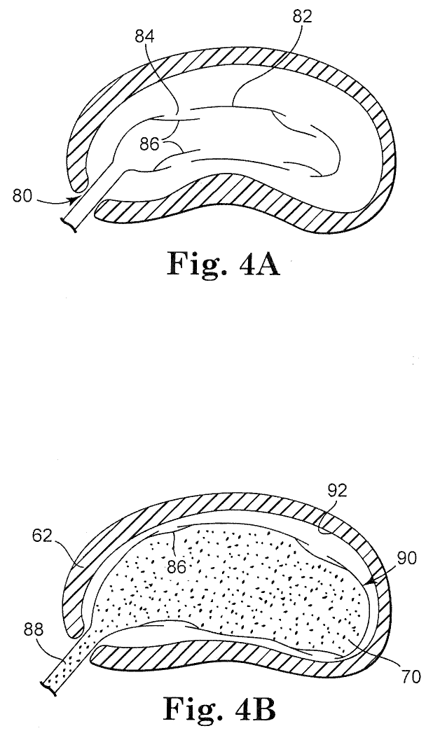

FIG. 4A illustrates an alternate mold assembly 80 in accordance with the present invention. Mold 82 includes a plurality of openings 84. The openings 84 can be any shape and a variety of sizes. Internal flaps 86 are located over the openings 84. As best illustrated in FIG. 4B, biomaterial 70 is delivered through lumen 88 to the mold 82. Pressure from the biomaterial 70 presses the flaps 86 against the openings 84, substantially sealing the biomaterial 70 within the mold 82.

In one embodiment, the flaps 86 permit any air or biomaterial in the mold 82 to be pushed out through the openings 84 during delivery of the biomaterial 70. In another embodiment, the flaps 86 to not completely seal the openings 84 until the mold 82 is substantially inflated and pressing against inner surface 92 of the annulus 62.

The flaps 86 can be constructed from the same or different material than the mold 82. In one embodiment, the flaps 86 are constructed from a radiopaque material that is easily visible using various imaging technologies. Prior to the delivery of the biomaterial 70, such as illustrated in FIG. 4A, the spacing between the flaps 86 indicates that the mold 82 is not inflated. After delivery of the biomaterial 70, such as illustrated in FIG. 4B, the spacing between the flaps 86 provides an indication of the shape and position of the intervertebral prosthesis 90 relative to the annulus 62. By strategically locating the openings 84 and flaps 86 around the outer surface of the mold 82, a series of images can be taken during delivery of the biomaterial 70 which will illustrate the prosthesis 90 during formation and provide reference points for evaluating whether the prosthesis 90 is properly positioned and fully inflated within the annulus 62.

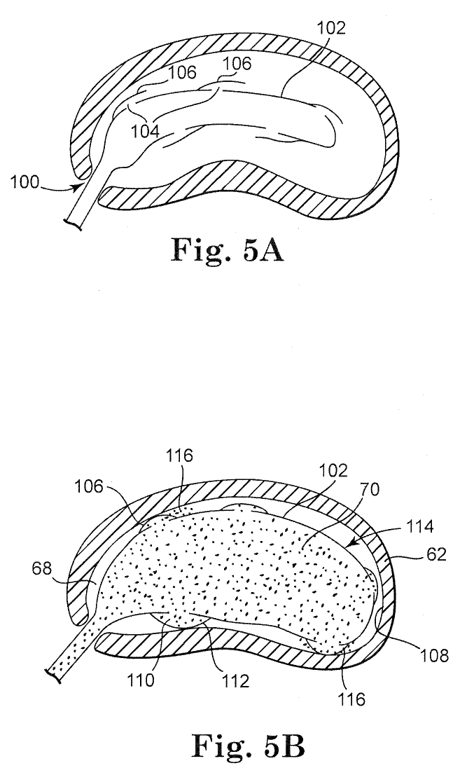

FIG. 5A illustrates an alternate mold assembly 100 in accordance with the present invention. Mold 102 includes a plurality of openings 104 with corresponding external flaps or valves 106. As best illustrated in FIG. 5B, delivery of the biomaterial 70 causes the mold 102 to inflate. When the mold 102 is substantially inflated, the flaps 106 are pressed against the openings 104 by interior surface 108 of the nuclear cavity 68.

In the illustrated embodiment, portion 110 of the biomaterial 70 forms a raised structure 112 over some or all of the openings 104. These raised structures serve to anchor the resulting prosthesis 114 in the nuclear cavity 68. Other examples of raised structures include barbs, spikes, hooks, and/or a high friction surface that can facilitate attachment to soft tissue and/or bone. Also illustrated in FIG. 5B, portion 116 of the biomaterial 70 optionally escapes from the mold 102 prior to the flaps 106 being pressed against the openings 104. The portion 116 of the biomaterial 70 serves to adhere the prosthesis 114 to the inner surface 108 of the annulus 62. Again, one or more of the mold 102, the flaps 106 may include radiopaque properties.

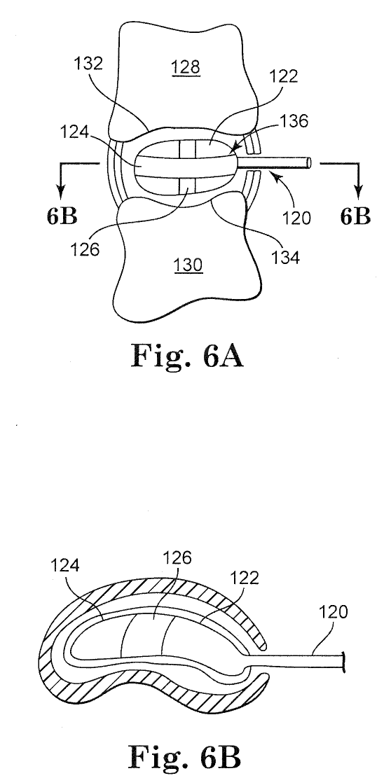

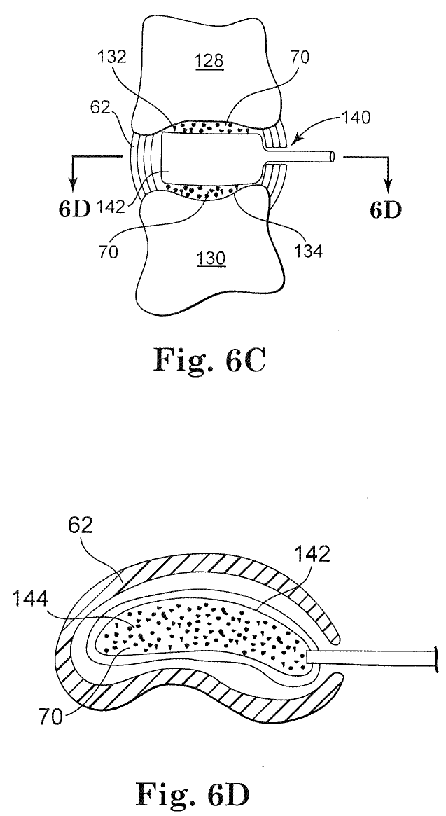

FIGS. 6A and 6B illustrate an alternate mold assembly 120 in accordance with the present invention. Mold 122 includes one or more reinforcing bands 124, 126. In the illustrated embodiment, reinforcing band 124 is attached to outer perimeter of the mold 122 and is positioned horizontally between adjacent vertebrae 128, 130. Reinforcing band 126 is oriented perpendicular to the band 124 and in the center of the mold 122 so as to be positioned opposite end plates 132, 134 of the opposing vertebrae 128, 130, respectively. In an alternate embodiment, one or both of the reinforcing bands 124, 126 can be located at the interior of the mold 122. The reinforcing bands 124, 126 can optionally be attached to the mold 122. In one embodiment, the reinforcing bands 124, 126 comprises thicker wall segments of the mold 122.

The band 124 preferably limits the amount of pressure the resulting prosthesis 136 places on the annular walls 62. A compressive force placed on the prosthesis 136 by the end plates 132, 134 is directed back towards the end plates, rather than horizontally into the annular wall 62. The band 126 preferably limits inflation of the mold 122 in the vertical direction. The band 126 can optionally be used to set a maximum disc height or separation between the adjacent vertebrae 128, 130 when the mold 122 is fully inflated.

In the illustrated embodiment, the bands 124, 126 are preferably radiopaque. As with the flaps 86, 106 of FIGS. 4 and 5, the bands 124, 126 provide an indication of the shape and position of the prosthesis 136 in the intervertebral disc space 138. As the biomaterial is delivered to the mold 122, the reinforcing bands 124, 126 are deployed and positioned in accordance with the requirements of the prosthesis 136. A series of images can be taken of the intervertebral disc space 138 to map the progress of the prosthesis formation. Because the size and width of the bands 124, 126 are known prior to the procedure, the resulting images provide an accurate picture of the position of the prosthesis 136 relative to the vertebrae 128, 130.

FIGS. 6C and 6D illustrate an alternate mold assembly 140 in accordance with the present invention. The reinforcing band 142 is preferably positioned horizontally between adjacent vertebrae 128, 130. In the illustrated embodiment, the reinforcing band 142 also serves as a mold for retaining at least a portion of the biomaterial 70. The annulus wall 62 may also act to retain the biomaterial 70 in the intervertebral disc space.

In one embodiment, the reinforcing band 142 preferably extends to the endplates 132, 134 so that the biomaterial 70 is substantially retained in center region 144 formed by the reinforcing band 142. In the embodiment of FIG. 6C, the biomaterial 70 extends above and below the reinforcing band 142 to engage with the endplates 132, 134. As best illustrated in FIG. 6D, the reinforcing band 142 is open at the top and bottom. In some embodiments, the biomaterial 70 may flow around the outside perimeter of the reinforcing band 142.

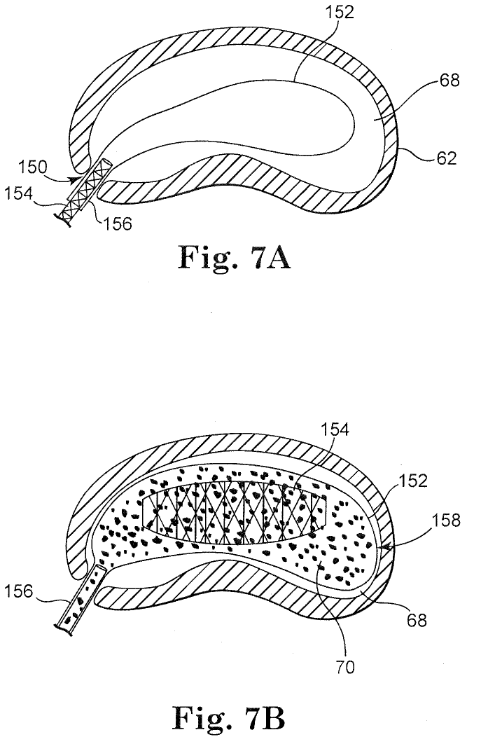

FIGS. 7A and 7B illustrate an alternate mold assembly 150 in accordance with the present invention. Mold 152 is positioned in nuclear cavity 68 of the annulus 62. Reinforcing structure or scaffolding 154 configured in a compressed state is delivered into the mold 152 through delivery lumen 156.

As best illustrated in FIG. 7B, once the reinforcing structure 154 is released from the delivery lumen 156, it assumes its original expanded shape within the nuclear cavity 68. The biomaterial 70 is delivered to the mold 152, where it flows into and around the reinforcing structure 154, creating a reinforced prosthesis 158. In an alternate embodiment, the reinforcing structure is deployed by the pressure of the biomaterial 70 being delivered into the mold 152.

In the illustrated embodiment, the reinforcing structure 154 is a mesh woven to form a generally tubular structure. The mesh 154 can be constructed from a variety of metal, polymeric, biologic, and composite materials suitable for implantation in the human body. In one embodiment, the mesh operates primarily as a tension member within the prosthesis 158. Alternatively, the reinforcing structure 154 is configured to act as both a tension and compression member within the prosthesis 158.

In another embodiment, the reinforcing structure 154, or portions thereof, are constructed from a radiopaque material. In the expanded configuration illustrated in FIG. 7B, the radiopaque elements of the reinforcing structure 154 provide a grid or measuring device that is readily visible using conventional imaging techniques. The reinforcing structure 154 thus provides a way to determine the shape, volume, dimensions, and position of the prosthesis 158 in the annular cavity 68. The reinforcing structure 162 can also serve to seal the opening of the mold 152 to the lumen, preventing biomaterial from leaving the mold.

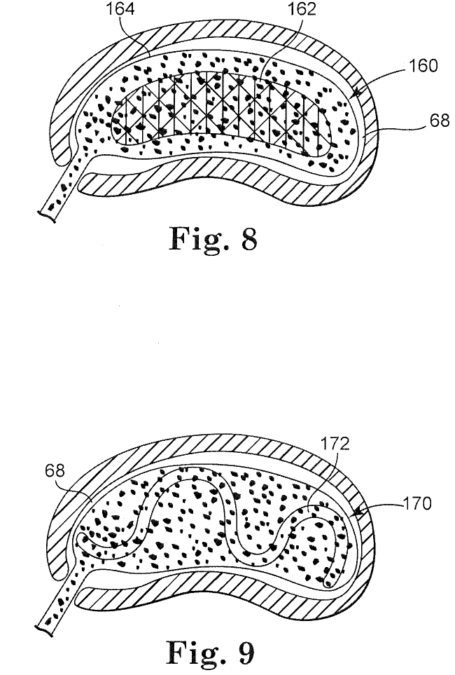

FIG. 8 illustrates an alternate prosthesis 160 with an internal reinforcing structure 162 having a shape generally corresponding to the nuclear cavity 68. As illustrated in FIG. 7, the reinforcing structure 162 is compressed within the delivery lumen 156 (see FIG. 7A) and delivered into mold 164 located in the nuclear cavity 68. Once in the expanded configuration illustrated in FIG. 8, the reinforcing structure 162 can operate as a tension and/or compression member within the prosthesis 160.

FIG. 9 illustrates an alternate prosthesis 170 in accordance with the present invention. Reinforcing structure 172 is again positioned in the nuclear cavity 68 in a compressed configuration through a delivery lumen 156 (see FIG. 7A). The reinforcing structure 172 is preferably constructed of a shape memory alloy (SMA), such as the nickel-titanium alloy Nitinol or of an elastic memory polymer that assumes a predetermined shape once released from the delivery lumen 156 or once a certain temperature is reached, such as for example the heat of the body. In the preferred embodiment, the reinforcing structure 172 has radiopaque properties which can be used to facilitate imaging of the prosthesis 170.

In another embodiment, the reinforcing structure 172 is a mold configured with a coil shape. When inflatable with biomaterial 70, the mold forms a coil-shaped reinforcing structure. Additional biomaterial 70 is preferably delivered around the coil structure 172.

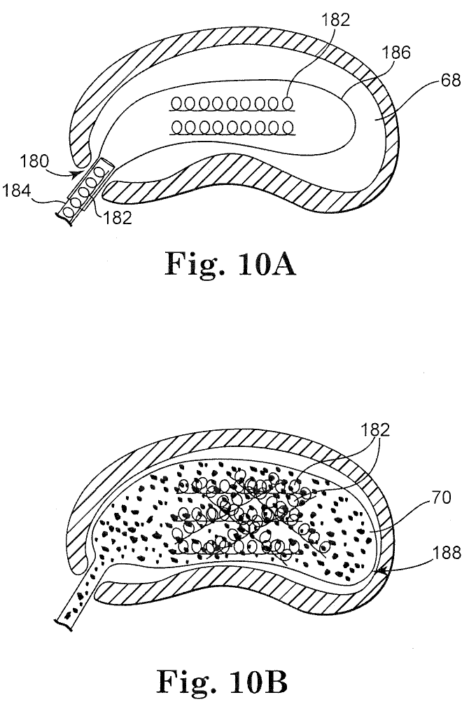

FIGS. 10A and 10B illustrate an alternate mold assembly 180 in accordance with the present invention. A plurality of discrete helical reinforcing structures 182 are delivered through a delivery lumen 184 into mold 186. As best illustrated in FIG. 10B, the helical reinforcing structures 182 intertwine and become entangled within the annular cavity 68. In one embodiment, the helical reinforcing structures 182 are rotated during insertion to facilitate engagement with the reinforcing structures 182 already in the mold 186.

Alternatively, these reinforcing structures 182 can be kinked strands, which when compressed have a generally longitudinal orientation to provide easy delivery through the lumen 184. Once inside the annular cavity, the reinforcing structures 182 are permitted to expand or reorient. The cross-sectional area of the reinforcing structures 182 in the expanded or reoriented state is preferably greater than the diameter of the lumen 184, so as to prevent ejection during delivery of the biomaterial 70. The reinforcing structures 182 can be delivered simultaneously with the mold 186 or after the mold 186 is located in the annular cavity 68.

The plurality of reinforcing structures 182 are preferably discrete structures that act randomly and can be positioned independently. The discrete reinforcing structures 182 of the present invention can be delivered sequentially and interlocked or interengaged in situ. Alternatively, groups of the reinforcing structures 182 can be delivered together.

In one embodiment, some or all of the reinforcing structures 182 are pre-attached to the inside of the mold 186, preferably in a compressed state. The reinforcing structures can be attached to the mold 186 during mold formation or after the mold is formed. As the mold 186 is inflated, whether with biomaterial 70 or simply inflated with a fluid during an evaluation step, the reinforcing structures 182 are stretched and/or released from the mold 186 and are permitted to resume their expanded shape. In one embodiment, some of the reinforcing structures 182 remain at least partially attached to the mold 186 after delivery of the biomaterial 70.

Once the biomaterial 70 is delivered and at least partially cured, the relative position of the reinforcing structures 182 is set. The reinforcing structures 182 can act as spring members to provide additional resistance to compression and as tension members within the prosthesis 188. Some or all of the helical reinforcing structures 182 preferably have radiopaque properties to facilitate imaging of the prosthesis 188.

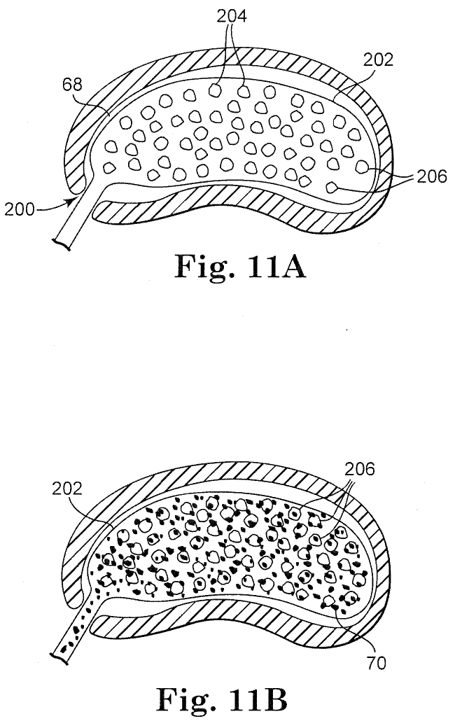

FIGS. 11A and 11B illustrate an alternate mold assembly 200 in accordance with the present invention. The mold 202 is located in the nuclear cavity 68. A plurality of reinforcing structures 204 are then delivered into the mold 202. Biomaterial 70 is then delivered to the mold 202, locking the reinforcing structures 204 in place. The reinforcing structures 204 typically arrange themselves randomly within the mold 202.

In the illustrated embodiment, the reinforcing structures 204 are a plurality of spherical members 206. The spherical members 206 flow and shift relative to each other within the mold 202. In one embodiment, the spherical members 206 are constructed from metal, ceramic, and/or polymeric materials. The spherical members 206 can also be a multi-layered structure, such as for example, a metal core with a polymeric outer layer.

In another embodiment, the spherical members 206 are hollow shells with openings into which the biomaterial 70 can flow. In this embodiment, the biomaterial 70 fills the hollow interior of the spherical members 206 and bond adjacent spherical members 206 to each other.

In one embodiment, the spherical members 206 have magnetic properties so they clump together within the mold 202 before the biomaterial 70 is delivered. Some or all of the spherical members 206 optionally have radiopaque properties.

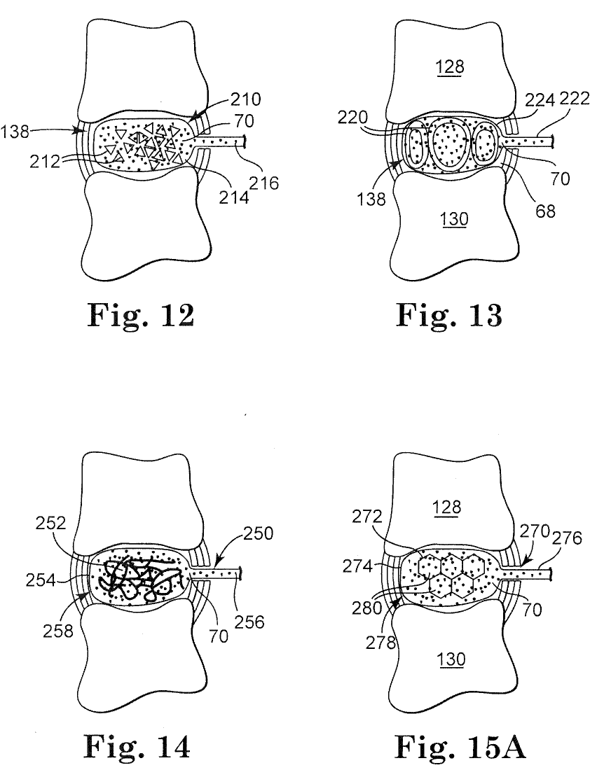

FIG. 12 is a side sectional view of an intervertebral disc space 138 containing prosthesis 210 in accordance with the present invention. A plurality of polyhedron reinforcing structures 212 are delivered into the mold 214 through lumen 216. For example, the reinforcing structure can be pyramidal, tetrahedrons, and the like. In one embodiment, the pyramidal reinforcing structures 212 have magnetic properties causing them to bind to each other within the mold 214. In another embodiment, the pyramidal reinforcing structures 212 include a plurality of holes or cavities into which the biomaterial 70 flows, securing the reinforcing structures 212 relative to each other and relative to the prosthesis 210.

FIG. 13 is a side sectional view of an intervertebral disc space 138 with prosthesis 224 having coiled or loop shaped reinforcing structures 220 in accordance with the present invention. The reinforcing structures 220 can be compressed for delivery through the lumen 222, and allowed to expand once inside the nuclear cavity 68. Biomaterial 70 is then injected to secure the relative position of the reinforcing structures 220 within the prosthesis 224.

The reinforcing structures 220 are preferably constructed from a spring metal that helps maintain the separation between the adjacent vertebrae 128, 130. In one embodiment, the reinforcing structures 220 are resilient and flex when loaded. In an alternate embodiment, the reinforcing structures 220 are substantially rigid in at least one direction, while being compliant in another direction to permit insertion through the lumen 222. The reinforcing structures 220 optionally define a minimum separation between the adjacent vertebrae 128, 130. The reinforcing structures 220 can operate as tension and/or compression members.

FIG. 14 is a side sectional view of an alternate mold assembly 250 in accordance with the present invention. A plurality of reinforcing fibers 252 are delivered into the mold 254 through lumen 256. The biomaterial 70 is then delivered and secures the relative position of the reinforcing fibers 252 within the mold 254. The reinforcing fibers 252 can be in the form of individual strands, coils, woven or non-woven webs, open cell foams, closed cell foams, combination of open and closed cell foams, scaffolds, cotton-ball fiber matrix, or a variety of other structures. The reinforcing fibers 252 can be constructed from metal, ceramic, polymeric materials, or composites thereof. The reinforcing fibers 252 can operate as tension and/or compression members within prosthesis 258.

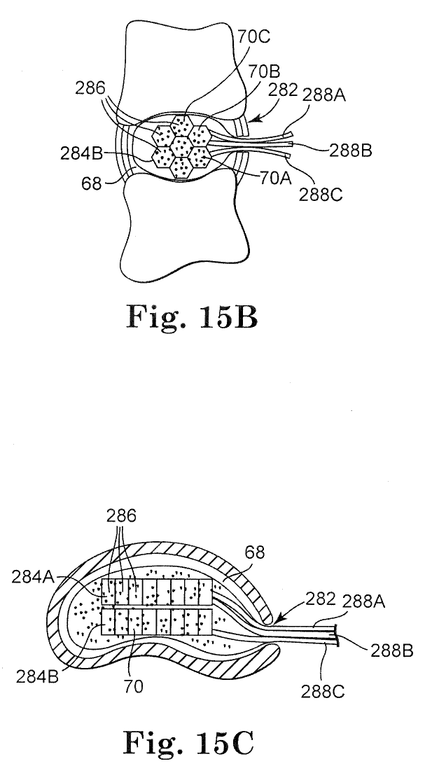

FIG. 15A is a side sectional view of an alternate mold assembly 270 in accordance with the present invention. A three-dimensional honeycomb structure 272 is compressed and delivered into the mold 274 through the lumen 276. Once in the expanded configuration, illustrated in FIG. 15A, the biomaterial 70 is delivered, fixing the honeycomb structure 272 in the illustrated configuration. In another embodiment, the delivery of the biomaterial expands or inflates the honeycomb structure 272.

The biomaterial 70 flows around and into the honeycomb structure 272 providing a highly resilient prosthesis 278. In one embodiment, the honeycomb structure 272 still retains its capacity to flex along with the biomaterial 70 when compressed by the adjacent vertebrae 128, 130. The honeycomb structure 272 can be constructed from a plurality of interconnected tension and/or compression members. In yet another embodiment, the honeycomb structure is an open cell foam.

In one embodiment, the honeycomb structure 272 has fluid flow devices, such as for example pores, holes of varying diameter or valves, interposed between at least some of the interconnected cavities 280. The fluid flow devices selectively controlling the flow of biomaterial 70 into at least some of the cavities 280 or filling the cavities 280 differentially, thus combining the different mechanical properties of the honeycomb structure 272 with the biomaterial 70 in an adaptable manner. The generally honeycomb structure 272 can optionally be combined with open or closed cell foam.

FIGS. 15B and 15C are side and top sectional views of the mold assembly 282 with a plurality of three-dimensional honeycomb structures 284A, 284B (referred to collectively as “284”) in accordance with the present invention. The honeycomb structures 284 are constructed so that the inflow of biomaterial 70 can be selectively directed to certain cavities 286. In alternate embodiments, more than two honeycomb structures 284A, 284B can optionally be used.

In one embodiment, holes interconnecting adjacent cavities 286 can be selectively opened or closed before the honeycomb structures 284 are inserted into the patient. In another embodiment, a plurality of lumens 288A, 288B, 288C, . . . (referred to collectively as “288”) are provided that are each connected to a different cavity 286. One or more of the lumens 288 can also be used to evacuate the annular cavity 68.

Selective delivery of the biomaterial 70 into the honeycomb structures 284 can be used to create a variety of predetermined internal shapes. Using a plurality of lumens 288 permits different biomaterials 70A, 70B, 70C, . . . to be delivered to different cavities 286 within the honeycomb structure 284. The biomaterials 70A, 70B, 70C, . . . can be selected based on a variety of properties, such as mechanical or biological properties, biodegradability, bioabsorbability, ability to delivery bioactive agents. As used herein, “bioactive agent” refers to cytokines and preparations with cytokines, microorganisms, plasmids, cultures of microorganisms, DNA-sequences, clone vectors, monoclonal and polyclonal antibodies, drugs, pH regulators, cells, enzymes, purified recombinant and natural proteins, growth factors, and the like.

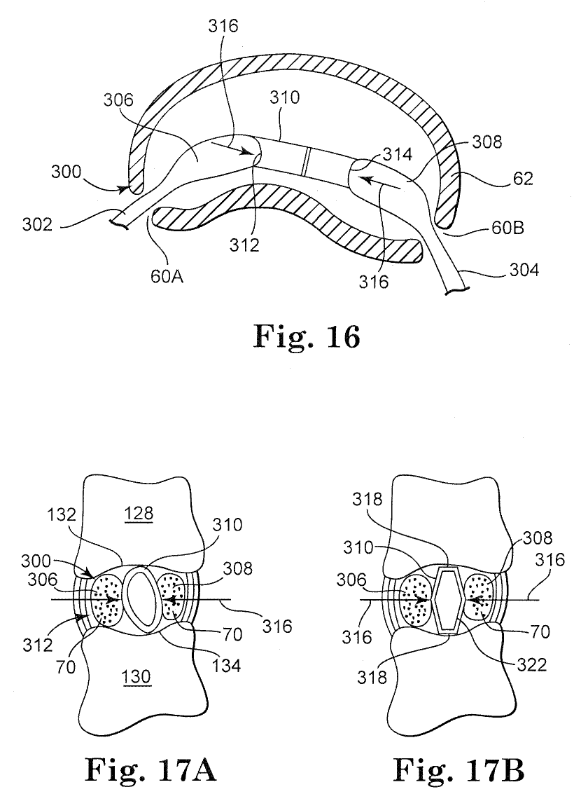

FIG. 16 illustrates an alternate mold assembly 300 in accordance with the present invention. In the illustrated embodiment, two annulotomies 60A, 60B are formed in the annulus 62. The mold assembly 300 is threaded through one of the annulotomies so that the lumens 302, 304 each protrude from annulotomies 60A, 60B, respectively. Lumen 302 is fluidly coupled to mold 306 while lumen 304 is fluidly coupled with mold 308. Reinforcing structure 310 is attached to molds 306, 308 at the locations 312, 314, respectively.

FIG. 17A is a side sectional view of the mold assembly 300 of FIG. 16 implanted between adjacent vertebrae 128, 130. Biomaterial 70 is delivered to the molds 306, 308, which applies opposing compressive forces 316 on the reinforcing structure 310. In the illustrated embodiment, the reinforcing structure 310 is a coil, loop, or bend (arc) of resilient material, such as a memory metal, spring metal, and the like. The resulting prosthesis 312 includes a pair of molds 306, 308 containing a cured biomaterial 70 holding the reinforcing structure 310 against adjacent end plates 132, 136 of the vertebrae 128, 130 respectively. The reinforcing structure can serve to resist compression, bending, tension, torsion, or a combination thereof, of the prosthesis 312 or to establish a minimum separation between the adjacent end plates 132, 134.

FIG. 17B is an alternate embodiment of the mold assembly 300 of FIG. 16. In the illustrated embodiment, reinforcing structure 310 includes a series of fold lines or hinges 318. Expansion of the molds 306, 308 with biomaterial 70 generates forces 316 that converts the generally flat reinforcing structure 310 (see FIG. 16) into the shaped reinforcing structure 322 illustrated in FIG. 17B. Alternatively, the hinge 318 could be facing the molds 306, 308 rather than the endplates. In the embodiments of FIGS. 17A and 17B, delivery of the biomaterial 70 deploys the reinforcing structure 310 to an expanded configuration.

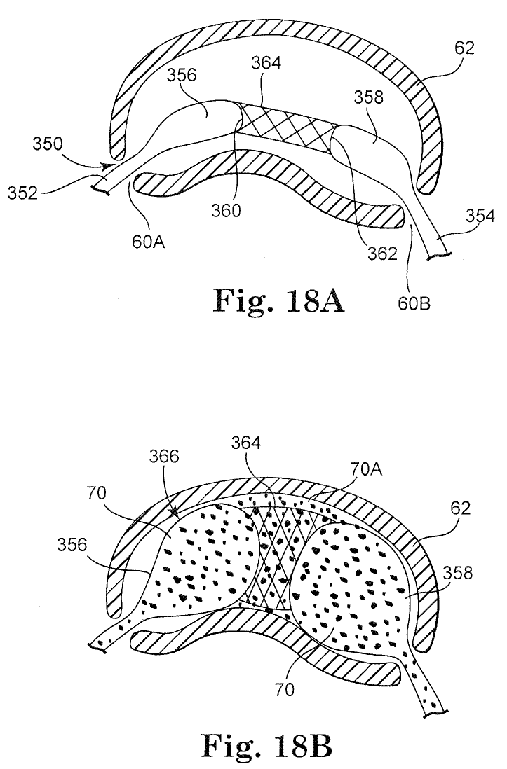

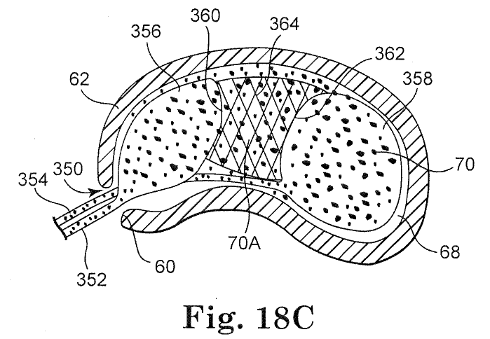

FIGS. 18A and 18B illustrate an alternate mold assembly 350 in accordance with the present invention. Lumens 352, 354 extend into the annulus 62 through different annulotomies 60A, 60B. Lumen 352 is fluidly coupled with mold 356 and lumen 354 is fluidly coupled with mold 358. Reinforcing mesh structure 364 is connected to the molds 356, 358 at locations 360, 362, respectively. As illustrated in FIG. 18B, biomaterial 70 is delivered to the molds 356, 358 causing the reinforcing structure 364 to be compressed and/or stretched within the nuclear cavity 68.

In one embodiment, additional biomaterial 70 can optionally be delivered into the nuclear cavity 68 proximate the reinforcing structure 364. In the illustrated embodiment, the same or a different biomaterial 70A flows around and into the reinforcing structure 364. The biomaterial 70A bonds the reinforcing structure 364 to the annulus 62. The resulting prosthesis 366 has three distinct regions of resiliency. The areas of varying resiliency can be tailored for implants that would be implanted via different surgical approaches, as well as various disease states. The reinforcing structure 364 optionally includes radiopaque properties. A series of images taken during delivery of the biomaterial 70 illustrates the expansion and position of the prosthesis 366 in the nuclear cavity 68.

FIG. 18C is an alternate configuration of the mold assembly 350 for use with mono-portal applications in accordance with the present invention. Lumens 352, 354 extend into the annulus 62 through a single annulotomy 60. Lumen 352 is fluidly coupled with mold 356 and lumen 354 is fluidly coupled with mold 358. Reinforcing mesh structure 364 is connected to the molds 356, 358 at locations 360, 362, respectively. As illustrated in FIG. 18B, delivery of the biomaterial 70 causing the reinforcing structure 364 to be compressed and/or stretched within the nuclear cavity 68. Additional biomaterial 70A can optionally be delivered into the nuclear cavity 68 proximate the reinforcing structure 364.

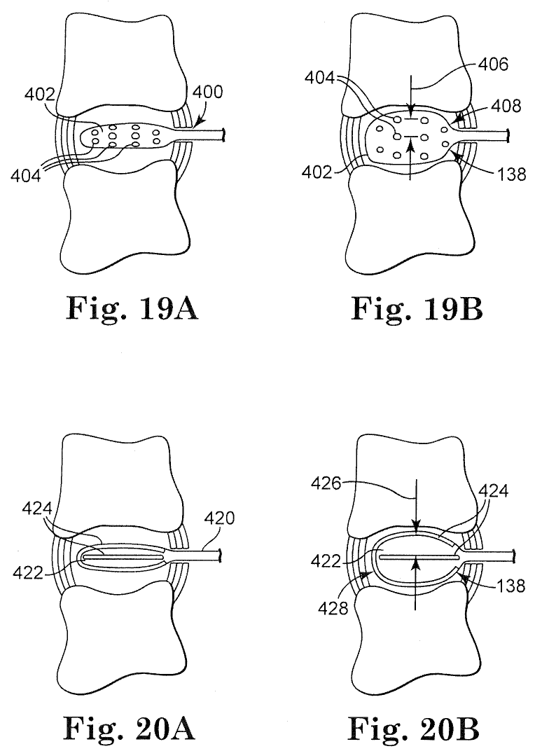

FIGS. 19A and 19B are side sectional views of mold assembly 400 in accordance with the present invention. The mold 402 includes a plurality of radiopaque markers 404. In the illustrated embodiment, the radiopaque markers 404 are arranged in a predetermined pattern around the perimeter of the mold 402. As best illustrated in FIG. 19B, once the mold 402 is inflated with the biomaterial, the spacing 406 between the adjacent radiopaque markers 404 increases. By imaging the intervertebral disc space 138 before, during and after delivery of the biomaterial 70, a series of images can be generated showing the change in the spacing between the radiopaque markers 404. Because the spacing between the radiopaque markers 404 is known prior to delivery of the biomaterial, it is possible to calculate the shape and position of the prosthesis 408 illustrated in FIG. 19B using conventional imaging procedures.

FIGS. 20A and 20B illustrate an alternate mold assembly 420 in accordance with the present invention. Mold 422 includes a plurality of radiopaque strips 424 located strategically around its perimeter. When the mold 422 is inflated with biomaterial, the spacing 426 between the radiopaque strips 424 changes, providing an easily imageable indication of the shape and position of the prosthesis 428 in the intervertebral disc space 138.

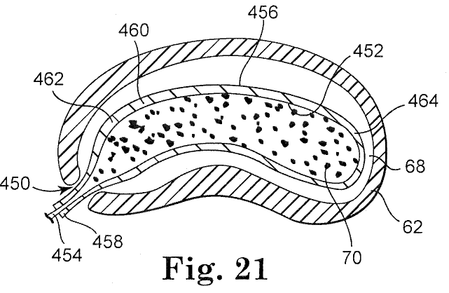

FIG. 21 illustrates an alternate mold assembly 450 in accordance with the present invention. Inner mold 452 is fluidly coupled to lumen 454. Outer mold 456 is fluidly coupled to lumen 458. Biomaterial is delivered through the lumen 454 into the inner mold 452. A radiopaque fluid is preferably delivered to the space 460 between the inner mold 452 and the outer mold 456.

In one embodiment, as the biomaterial 70 is delivered to the inner mold 452, the radiopaque material 462 located in the space 460 is expelled from the nuclear cavity 68 through the lumen 458. A series of images of the annulus 62 will show the progress of the biomaterial 70 expanding the inner mold 452 within the nuclear cavity 68 and the flow of the radiopaque fluid 462 out of the space 460 through the lumen 458.

In another embodiment, once the delivery of the biomaterial 70 is substantially completed and the radiopaque material 462 is expelled from the space 460, a biological material or bioactive agent is injected into the space 460 through the delivery lumen 458. In one embodiment, the outer mold 456 is sufficiently porous to permit the bioactive agent to be expelled into the annular cavity 68, preferably over a period of time. One of the molds 452, 456 optionally includes radiopaque properties. The mold 456 is preferably biodegradable or bioresorbable with a half life greater than the time required to expel the bioactive agents.

In another embodiment, one or more reinforcing structures 464, such as disclosed herein, is located in the space 460 between the inner and outer molds 452, 456. For example, the reinforcing structure 464 may be a woven or non-woven mesh impregnated with the bioactive agent. In another embodiment, the reinforcing structure 464 and the outer mold 456 are a single structure, such as a reinforcing mesh impregnated with the bioactive agent. In yet another embodiment, the outer mold 456 may be a stent-like structure, preferably coated with one or more bioactive agents.

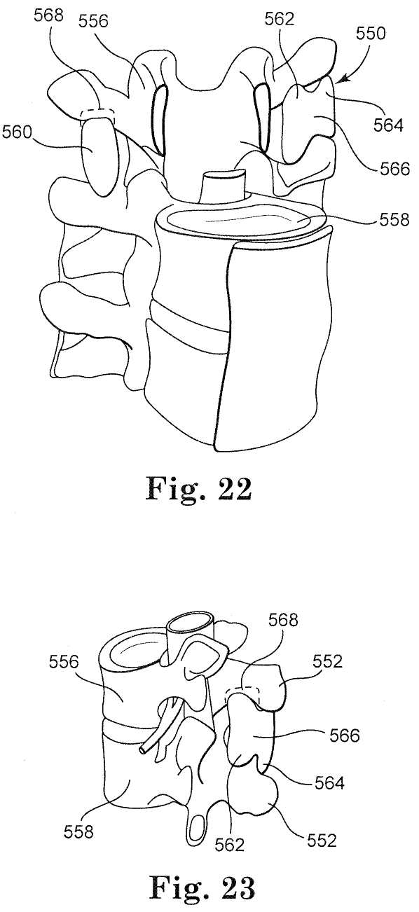

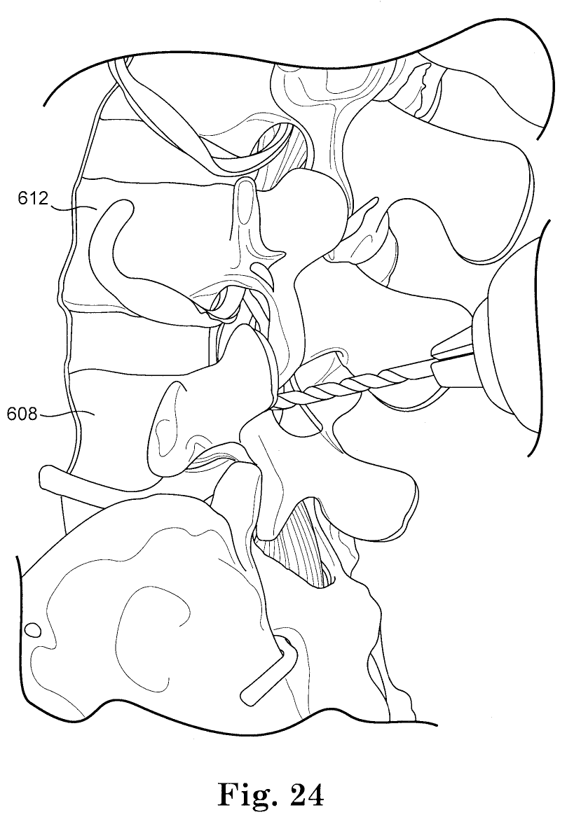

FIGS. 22 and 23 illustrate use of a mold assembly 550 to restore and/or maintain the separation between spinous process 552 and/or transverse processes 554 on adjacent vertebrae 556, 558 in according with the present method and apparatus. The mold assemblies and reinforcing structures disclosed herein can be used for this application. The mold assembly 550 may be used alone or in combination with an intervertebral mold assembly, such as discussed herein. The mold assembly 550 may also be used in combination with a variety of other spinal devices, including nucleus replacement, total disc replacement, interbody fusion, vertebral body replacement, pedicle screw fixation, facet replacement, facet fixation, and the like, examples of which are found in U.S. Pat. Nos. 4,636,217; 4,599,086; 5,192,327; 4,932,975; 5,458,638; 5,425,772; 5,306,309; 5,766,252; 5,534,031; 5,676,666; 5,954,722; 4,653,481; 5,005,562; 5,645,599; 5,674,296; 5,676,701; 5,507,816, which are hereby incorporated by reference.

The mold assembly 550 can also be used to separate the superior articulating process and inferior articulating process, more commonly referred to as the facet joint, on adjacent vertebrae. By inflating the mold 550 on one side of the sagittal plane greater than the mold 560 on the other side of the sagittal plane, the present system can be used to correct lateral curvature of the spine, such as for example scoliosis. Selective inflation of prosthetic devices is disclosed in U.S. application Ser. No. 12/014,560, entitled In Situ Adjustable Dynamic Intervertebral Implant, filed Oct. 24, 2007, which is hereby incorporated by reference.

In the illustrated embodiment, the mold 560 preferable includes extension 562, 564 that couple or engage with the spinous process or transverse processes 552, 554. Center portion 566 acts as a spacer to maintain the desired separation. In one embodiment, the mold assembly has an H-shaped or figure-8 shaped cross section to facilitate coupling with the various facets on the adjacent vertebral bodies. Attachment of the molds 550 or 560 to the spinous or transverse processes may be further facilitated using sutures, cables, ties, rivets, screws, clamps, sleeves, collars, adhesives, or the like. Any of the mold assemblies and reinforcing structures disclosed herein can be used with the mold assembly 550. In an alternate embodiment, the posterior elements are contoured 568 to enhance engagement with the molds 560.

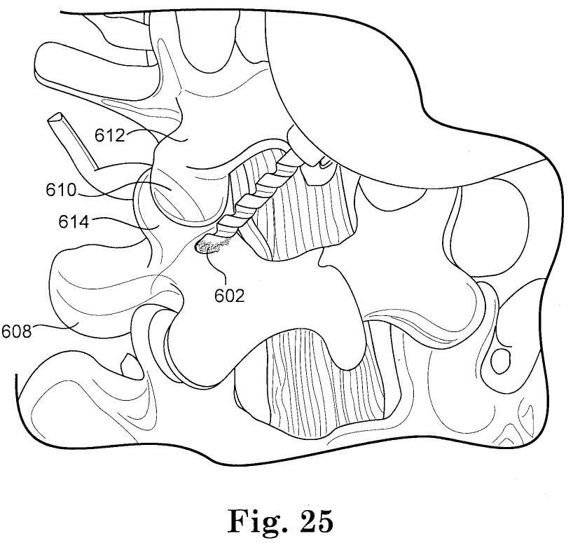

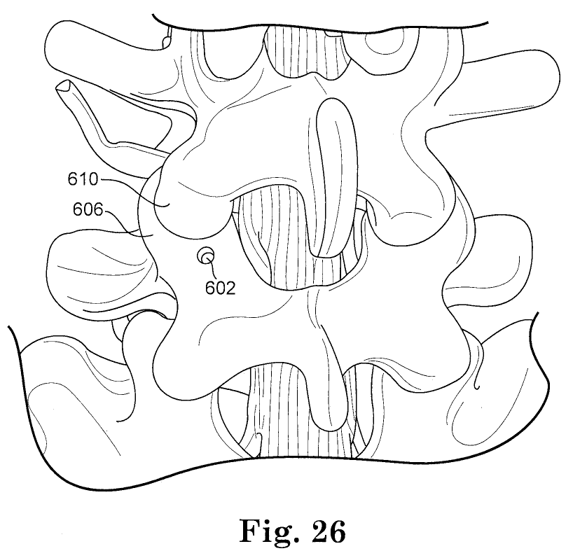

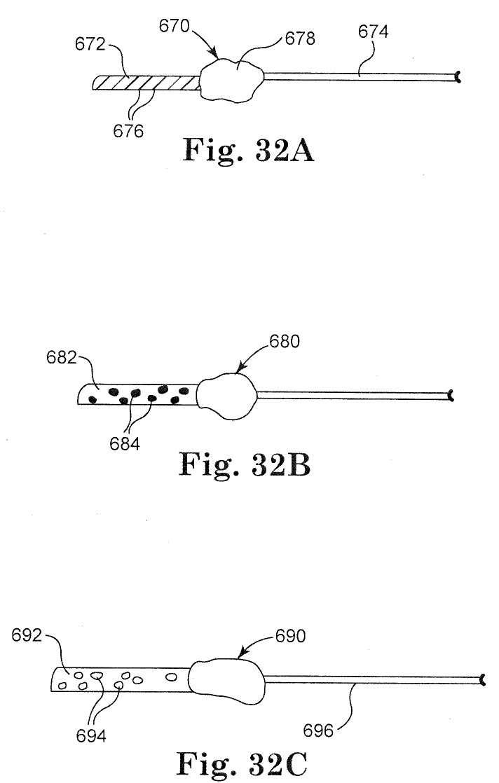

FIGS. 24 through 27 illustrate the use of a mold assembly 600 according to an embodiment of the present invention. The mold assembly 600 is located in a hole 602 in superior facet 614 of inferior vertebrae 608. The mold assemblies illustrated in FIGS. 32A-32C are particularly suited for this application.

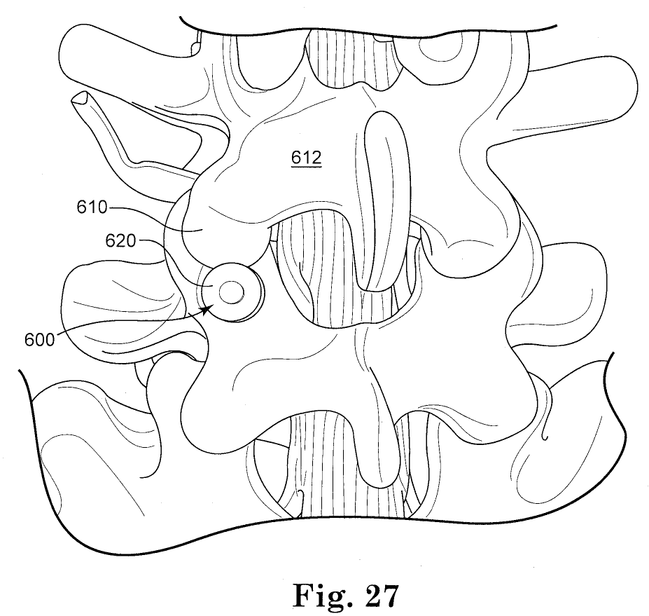

As best illustrated in FIG. 24, hole 602 is drilled into and through pedicle 606 of inferior vertebrae 608. The hole 602 is preferably drilled without violating the inferior facet 610 of the superior vertebrae 612. Mold assembly 600 is at least partially located in the hole 602 and inflated with biomaterial. As illustrated in FIG. 27, head or bumper 620 inflates and abuts against inferior articulating facet 610 of the superior vertebrae 612. An extension of the mold assembly 600 (see e.g., FIGS. 32A-32C) may optionally be used to secure the mold assembly to the superior facet 614 of the inferior vertebrae 608.

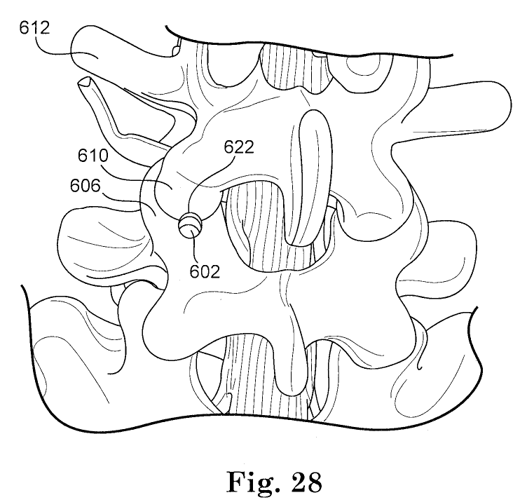

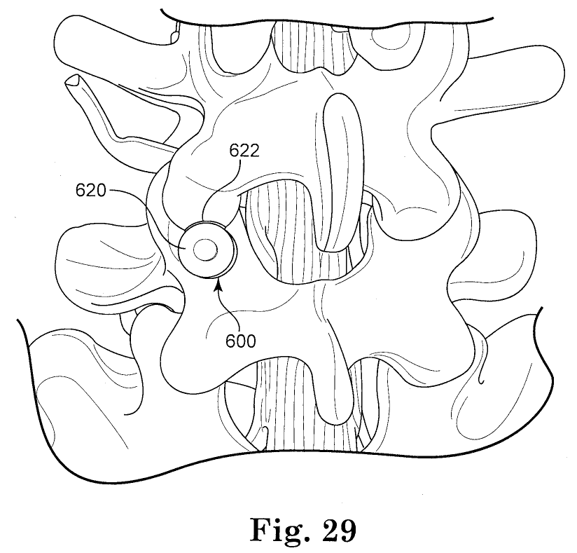

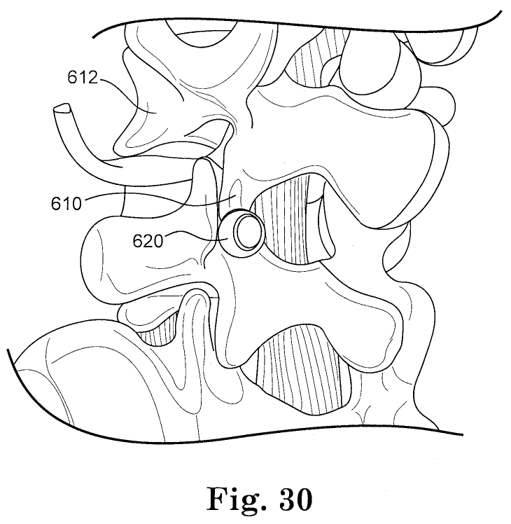

In an alternate embodiment illustrated in FIGS. 28 through 30, an edge of the inferior articulating facet 610 of the superior vertebrae 612 is contoured to engage with the head 620. When the mold assembly 600 is located in the hole 602, head 620 engages with contoured surface 622 of the inferior facet 610. The contoured surface 622 preferably corresponds with an external surface of the head 620. In another embodiment, the contoured surface 622 may be formed on the surface of the superior articulating facet 614 of the inferior vertebrae 608 so that the head 620 engages with that surface.

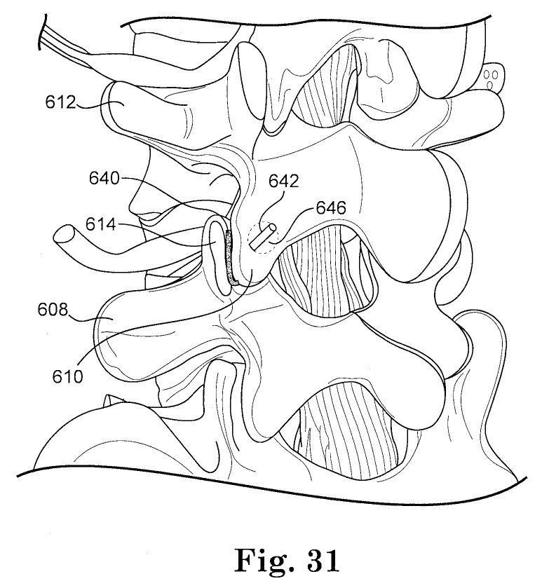

FIG. 31 illustrates another embodiment in which a prosthesis 640 is formed in situ between the inferior articulating facet 610 of the superior vertebrae 612 and the superior articulating facet 614 of the inferior vertebrae 608. In the illustrated embodiment, the prosthesis 640 is inserted through a hole in the inferior articulating facet 610 of the superior vertebrae 612.

Catheter segment 642 can optionally be used to anchor the prosthesis 640 in place. For example, a fastener 646 can be attached to the catheter segment 642. Alternatively, the catheter segment 642 can be deformed 646 to have a cross section larger than a cross section of the hole through it was introduced. In another embodiment, catheter segment 642 is constructed from an elastically or plastically deformable material, so that the pressure of the biomaterial forms a bulbous portion 646 to lock the prosthesis 640 in place.

When the prosthesis 640 is inflated with the biomaterial it pushes the inferior articulating facet 610 and associated superior vertebrae 612 upwards and distracts the foramen. In another embodiment, the mold assembly 550 can be used in combination with the mold assemblies 600 and/or 640.

FIG. 32A illustrates an alternate mold assembly 670 suitable for use in any of the embodiments of FIGS. 24-31. The mold assembly 670 includes head or bumper 678 and extension 672 adapted to engage with the hole 602. The extension 672 can be a portion of lumen 674 used to deliver biomaterial to the mold assembly 670 or a separate fastening structure. In the illustrated embodiment, the extension 672 includes threads 676. As illustrated in FIG. 31, a portion of the lumen 674 can be used to secure the mold assembly 670.

After the head 678 and/or extension 672 are filled with biomaterial, the lumen 674 is typically cut at or near the head 678 and removed from the patient. If used in the embodiment of FIG. 31, the extension 672 is omitted or shorter than illustrated in FIG. 32A, and a portion of the lumen 674 is optionally left attached to the head 678 to anchor the device to the inferior articulating facet 610 of the superior vertebrae 612.

FIG. 32B illustrates mold assembly 680 with an extension 682 having a texture surface 684. FIG. 32C illustrates mold assembly 690 with extension 692 with a plurality of openings 694. The extension 692 is fluidly coupled to lumen 696 so that a portion of the biomaterial is extruded through the openings 694 to help secure the mold 690 to the posterior elements.

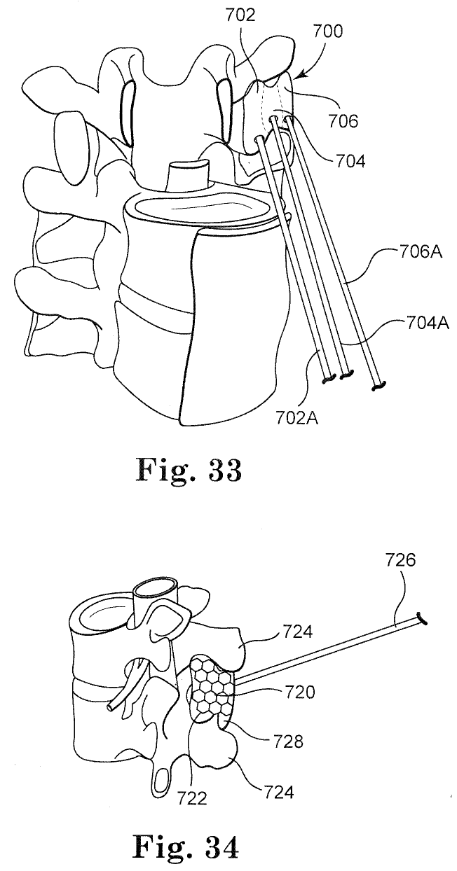

FIG. 33 illustrates an alternate mold 700 with a plurality of compartments 702, 704, 706 each preferably having a separate lumen 702A, 704A, 706A. The separate lumens 702A, 704A, 706A permit selective and differential inflation of the compartments 702, 704, 706. The mold 700 provides the surgeon additional control and adaptability to locally manipulate tissue, such as for example to distract in stenosis or to buffer in facet arthrosis. The manipulation of tissue can be done by inflating the mold 700 before injection of the biomaterial or during injection of the biomaterial.

In an alternate embodiment, the mold 700 can be formed to inflate in a predetermined shape, such as for example the shape illustrated in FIG. 28. This embodiment can be operated with a single lumen.

FIG. 34 illustrates a reinforcing structure 720 used to prepare an implant site 722 in according with an embodiment of the present invention. The reinforcing structure is assembled or expanded in-situ to manipulate tissue at the implant site 722. When the posterior elements 724 are in the desired configuration, biomaterial is delivered through lumen 726 to fix the reinforcing structure 720 in place. In one embodiment, the reinforcing structure 720 expands in response to injection of the biomaterial.

In the illustrated embodiment, the reinforcing structure 720 is located inside mold 728, although the reinforcing structure 720 may be used without the mold 728. The biomaterial preferably penetrates the reinforcing structure 720 and inflates the mold 728 to secure the assembly to the posterior elements 724. In the illustrate embodiment, the reinforcing structure 720 operates as both a surgical instrument to prepare the implant site 722 and as a mold to retain biomaterial.

Any of the embodiments disclosed herein can be used in combination with an evaluation mold to determine location of the prosthesis, size of the prosthesis, displacement of the posterior spinal elements, and the like. Use of such an evaluation mold is disclosed in commonly assigned U.S. patent application Ser. No. 10/984,493, entitled Multi-Stage Biomaterial Injection System for Spinal Implants, which is incorporated by reference.

Any of the features disclosed herein can be combined with each other and/or with features disclosed in commonly assigned U.S. patent application Ser. No. 11/268,786, entitled Multi-Lumen Mold for Intervertebral Prosthesis and Method of Using Same, filed Nov. 8, 2005, which is hereby incorporated by reference. Any of the molds and/or lumens disclosed herein can optionally be constructed from biodegradable or bioresorbable materials. The lumens disclosed herein can be constructed from a rigid, semi-rigid, or pliable high tensile strength material. The various components of the mold assemblies disclosed herein may be attached using a variety of techniques, such as adhesives, solvent bonding, mechanical deformation, mechanical interlock, or a variety of other techniques.

The mold assembly of the present invention is preferably inserted into the nuclear cavity 68 through a catheter, such as illustrated in commonly assigned U.S. patent application Ser. No. 11/268,876 entitled Catheter Holder for Spinal Implants, filed Nov. 8, 2005, which is hereby incorporated by reference.

Various methods of performing the nuclectomy are disclosed in commonly assigned U.S. patent Ser. No. 11/304,053 entitled Total Nucleus Replacement Method, filed on Dec. 15, 2005, which is incorporated by reference. Disclosure related to evaluating the nuclectomy or the annulus and delivering the biomaterial 70 are found in commonly assigned U.S. patent application Ser. No. 10/984,493, entitled Multi-Stage Biomaterial Injection System for Spinal Implants, filed Nov. 9, 2004, which is incorporated by reference. Various implant procedures and biomaterials related to intervertebral disc replacement suitable for use with the present multi-lumen mold are disclosed in U.S. Pat. Nos. 5,556,429 (Felt); 6,306,177 (Felt, et al.); 6,248,131 (Felt, et al.); 5,795,353 (Felt); 6,079,868 (Rydell); 6,443,988 (Felt, et al.); 6,140,452 (Felt, et al.); 5,888,220 (Felt, et al.); 6,224,630 (Bao, et al.), and U.S. patent application Ser. Nos. 10/365,868 and 10/365,842, all of which are hereby incorporated by reference. The present mold assemblies can also be used with the method of implanting a prosthetic nucleus disclosed in a commonly assigned U.S. patent application Ser. No. 11/268,856, entitled Lordosis Creating Nucleus Replacement Method and Apparatus, filed on Nov. 8, 2005, which are incorporated herein by reference.

The mold assemblies and methods of the present invention can also be used to repair other joints within the spine such as the facet joints, as well as other joints of the body, including diarthroidal and amphiarthroidal joints. Examples of suitable diarthroidal joints include the ginglymus (a hinge joint, as in the interphalangeal joints and the joint between the humerus and the ulna); throchoides (a pivot joint, as in superior radio-ulnar articulation and atlanto-axial joint); condyloid (ovoid head with elliptical cavity, as in the wrist joint); reciprocal reception (saddle joint formed of convex and concave surfaces, as in the carpo-metacarpal joint of the thumb); enarthrosis (ball and socket joint, as in the hip and shoulder joints) and arthrodia (gliding joint, as in the carpal and tarsal articulations).

The present mold apparatus can also be used for a variety of other procedures, including those listed above. The present mold assembly can also be used to modify the interspinous or transverse process space. The mold can operate as a spacer/distractor between the inferior and superior spinous processes, thus creating a local distraction and kyphosis if desired. The theory behind these implants is that they expand the intervertebral foramen and thereby relieve pressure on the nerve root and spinal cord. The present injectable prosthesis is adapted to the individual anatomy and clinical situation of the patient, without the need for multiple implant sizes

Patents and patent applications disclosed herein, including those cited in the Background of the Invention, are hereby incorporated by reference. Other embodiments of the invention are possible. Many of the features of the various embodiments can be combined with features from other embodiments. For example, any of the securing mechanisms disclosed herein can be combined with any of the multi-lumen molds. It is to be understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. A system for the in situ formation of prostheses between adjacent vertebrae of a patient, the system comprising:
a first mold adapted to be located in an intervertebral disc space between the adjacent vertebrae;
at least a second mold adapted to be positioned between adjacent posterior elements;
lumens fluidly coupled to each of the molds; and
one or more in situ curable biomaterials adapted to be delivered through the lumens to the molds, the at least partially cured biomaterial and the molds cooperating to maintain a desired alignment of the adjacent vertebrae and the posterior elements.

2. The system of claim 1 wherein the second mold is adapted to be positioned between posterior elements on one side of a sagittal plane of the patient and a third mold adapted to be positioned between posterior elements on an opposite side of the sagittal plane.

3. The system of claim 2 wherein the second mold displaces the posterior elements a greater amount than the third mold.

4. The system of claim 1 comprising one or more discrete reinforcing structures located in at least one of the molds.

5. The system of claim 1 comprising a valve fluidly coupling the lumens to at least one of the molds.

6. The system of claim 1 wherein at least a portion of one of the molds comprises a porous structure.

7. The system of claim 1 wherein at least a portion of one of the molds comprises a biodegradable material.

8. The system of claim 1 wherein at least one of the molds comprises a predetermined shape.

9. The system of claim 1 wherein the second mold comprises a center portion with a plurality of extensions adapted to engage the posterior elements.

10. The system of claim 1 wherein at least one of the molds includes an exterior surface adapted to facilitate tissue in-growth.

11. The system of claim 1 wherein at least one of the molds includes an exterior surface comprising a bioactive agent.

12. The system of claim 1 wherein the second mold includes an exterior surface textured to grip the posterior elements.

13. The system of claim 1 wherein the molds and the biomaterial are adapted to be delivered using minimally invasive techniques.

14. The assembly of claim 1 wherein the lumens are releasably coupled to the molds.

15. The assembly of claim 1 comprising a separate source of biomaterial fluidly coupled to each lumen.

16. The assembly of claim 1 wherein the mold comprises an extension structured adapted to engage with the posterior elements.

17. The assembly of claim 16 wherein the extension comprises one or more of a portion of the lumen, a threaded member, or a textured surface.

18. The assembly of claim 1 wherein the mold comprises an extension with a plurality of openings, the extension being fluidly coupled to the lumen.

19. The assembly of claim 1 comprising an evaluation mold adapted to be located in one or more of the intervertebral disc space or between the adjacent posterior elements.

20. The assembly of claim 1 wherein the second mold comprises an extension and a head portion.

21. An apparatus for the in-situ formation of a prosthesis between adjacent posterior elements of the spine, the apparatus comprising:
a mold adapted to be positioned between the adjacent posterior elements, the mold including at least one interior cavity adapted to receive a flowable, curable biomaterial;
at least one lumen fluidly coupled to the mold;
an extension attached to the mold adapted to engage with the posterior elements;
a valve assembly releasably coupling the lumen to the mold;
a flowable, curable biomaterial adapted to be delivered through the at least one lumen to the mold; and
at least one biomaterial delivery apparatus adapted to deliver the biomaterial through the lumen to expand the mold while the mold is located between the adjacent posterior elements, wherein the at least partially cured biomaterial and the mold cooperate to maintain a desired alignment between the posterior elements.

22. The apparatus of claim 21 wherein at least a portion of the mold comprises a porous structure.

23. The apparatus of claim 21 wherein at least a portion of the mold comprises a biodegradable material.

24. The apparatus of claim 21 comprising at least one reinforcing structure located in the mold.

25. The apparatus of claim 21 wherein the mold includes an exterior surface adapted to facilitate tissue in-growth.

26. The apparatus of claim 21 wherein the mold includes an exterior surface comprising a bioactive agent.

27. The apparatus of claim 21 wherein the mold includes an exterior surface textured to grip the posterior elements.

28. The apparatus of claim 21 wherein the mold comprises a porous structure containing a bioactive agent.

29. The apparatus of claim 21 wherein the mold and the biomaterial are adapted to be delivered using minimally invasive techniques.

30. A method for the in-situ formation of prostheses between adjacent vertebrae of a patient, the method comprising the steps of:
positioning a first mold in an intervertebral disc space between the adjacent vertebrae;
positioning at least a second mold between adjacent posterior elements;
fluidly coupling at least one lumen to each of the molds;
delivering a flowable, curable biomaterial through the lumens to the first and second molds; and
at least partially curing the biomaterial, the at least partially cured biomaterial maintaining a desired alignment of the adjacent vertebrae and the posterior elements.

31. The method of claim 30 comprising:
positioning the second mold between posterior elements on one side of a sagittal plane of the patient; and
positioning a third mold between posterior elements on an opposite side of the sagittal plane.

32. The method of claim 31 wherein the second mold displaces the posterior elements a greater amount than the third mold.

33. The method of claim 30 comprising the step of contouring the posterior elements to enhance engagement with the second mold.

34. The method of claim 30 comprising delivering the biomaterial to the first mold before delivering it to the second mold.

35. The method of claim 30 comprising the step of:
positioning an evaluation mold in the intervertebral disc space;
delivering a fluid to the evaluation mold so that the mold substantially fills the intervertebral disc space;
estimating the quantity of flowable, curable biomaterial for the first mold based on the quantity of fluid delivered to the evaluation mold.

36. A method for the in-situ formation of prostheses acting on adjacent vertebrae of a patient, the method comprising the steps of:
drilling a hole in a superior facet of an inferior vertebrae of the adjacent vertebrae;
locating a mold assembly in the hole;
fluidly coupling at least one lumen to each of the molds;
delivering a flowable, curable biomaterial through the lumens to the mold assembly;
inflating the mold assembly to create a bumper on an upper surface of the superior facet adjacent to an inferior facet of the superior vertebrae of the adjacent vertebrae;
at least partially curing the biomaterial, the at least partially cured biomaterial acting as a stop on movement of the inferior facet on the superior vertebrae relative to the superior facet on the inferior vertebrae.

37. The method of claim 36 comprising the steps of:
contouring at least one of an edge of the inferior facet on the superior vertebrae and an edge of the superior facet on the inferior vertebrae; and
engaging the contoured surface with the bumper.

38. The method of claim 36 comprising the step of inflating the mold assembly to create a spacer located between the inferior facet of the superior vertebrae and the superior facet of the inferior vertebrae.

39. A method for the in-situ formation of prostheses acting on adjacent vertebrae of a patient, the method comprising the steps of:
drilling a hole in a superior facet of an inferior vertebrae of the adjacent vertebrae;
locating a mold assembly in the hole;
fluidly coupling at least one lumen to each of the molds;
delivering a flowable, curable biomaterial through the lumens to the mold assembly;
inflating the mold assembly to create a spacer between an upper surface of the superior facet adjacent to an inferior facet of the superior vertebrae of the adjacent vertebrae;
at least partially curing the biomaterial, the at least partially cured biomaterial acting as a spacer between the inferior facet on the superior vertebrae and the superior facet on the inferior vertebrae.

40. The method of claim 39 comprising the step of anchoring a portion of the lumen to an upper surface of the inferior facet on the superior vertebrae.
